(19) United States
(12) Patent Application Publication
(10) Pub. No.: US 2004/0072302 A1
(43) Pub. Date: Apr. 15, 2004

(54) High throughput production of antibodies to genomic derived proteins

(76) Inventors: Paul DiTullio, Northboro, MA (US); Kathleen M. Hehir, Grafton, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/619,856

(22) Filed: Jul. 15, 2003

Related U.S. Application Data
(60) Provisional application No. 60/397,059, filed on Jul. 18, 2002.

(51) Int. Cl. C12P 21/04

(52) U.S. Cl. 435/70.21

(57) ABSTRACT

The invention features a method of producing an antibody to a target antigen in an animal by contacting said animal with a genetically-matched cell containing a heterologous nucleic acid encoding the target antigen.

### RELATED APPLICATIONS

This application claims priority to provisional patent application Ser. No. 60/397,059, filed on Jul. 18, 2002, the entire contents of which are hereby incorporated by reference.

### FIELD OF THE INVENTION

The invention relates to methods of producing antibodies.

### BACKGROUND OF THE INVENTION

Antibodies are useful tools in basic research, drug development, and the fight against diseases both as a diagnostic reagent and therapeutic. A classical approach to producing an antibody was to challenge an animal with an immunogen, which, in many cases was a mixture of many different proteins, to elicit an immune response. The antibody could then be harvested directly from the blood as a polyclonal antibody or the spleen cells used to produce a hybridoma-secreting a monoclonal antibody. A major problem with this approach is that the antibody response was often non-specific, because a mixture of proteins was injected as a source of antigen.

With the advent of recombinant DNA technology, some of these problems were overcome since the protein of interest could be produced using a cloned gene or cDNA for use as an immunogen. In many cases, however, this approach failed because the protein could not be produced in sufficient quantity, the desired antibody was not generated, or sufficient information was not available about the protein or gene. The sequencing of the human genome and development of rapid gene isolation techniques has now compounded the problem through the identification/isolation of thousands of genes with unknown function.

### SUMMARY OF THE INVENTION

The invention features a method for producing an antibody to a protein directly from a nucleic acid such as a gene or cDNA. The method of producing an antibody to a target antigen in an animal (e.g., a mammal or a member of an avian species such as a chicken) is carried out by contacting the animal with a genetically-matched cell containing a heterologous nucleic acid. The term “avian” refers to any avian species, including but not limited to, chicken, turkey, duck, goose, quail, and pheasant. The genetically-matched cell is identical at genetic loci encoding histocompatibility antigens, e.g., the cell is syngeneic or genetically identical with respect to the animal into which the immunogen cell (or cell product) is to be introduced. The heterologous nucleic acid encodes the target antigen. For example, the heterologous nucleic acid is a cDNA or fragment thereof.

The method includes a step of producing an immortalized cell line from the species of interest or obtaining one commercially, cloning the gene or cDNA into an expression vector containing a selectable marker, transfecting the expression vector into the immortalized cell line, selecting for the transfected cells, and immunizing the animal with the culture media in which the cells were incubated, a cell lysate from the transfected cells, or the intact cells themselves. Following immunization of an animal, the presence of antibody is tested using media or a lysate from the transfected cell line as a source of target antigen. The function or identity of the target antigen is known or unknown.

The cells used for immunization are syngeneic (genetically identical) or genetically very closely matched to minimize production of irrelevant antibodies (e.g., antibodies produced to proteins other than those encoded by the transfected nucleic acid). A key feature of the invention is the use of syngeneic cell line for expression of the target protein antigen. The animal and the immunogen cell are syngeneic at major histocompatibility complex (MHC) loci. For example, they are syngeneic at MHC class I loci, class II loci, or both class I and class II loci. When the immunogen cell is syngeneic, an antibody response is directed to the antigen encoded by the exogenous DNA, because all other proteins in the cell are viewed by the animal as self. The cell expresses a gene product encoded by the nucleic acid. Preferably, the gene product or a fragment thereof is expressed on a surface of said cell. In addition, the protein is presented in its native configuration and properly modified post-translationally, which results in the production of higher quality antibodies. For example, the antibodies have a high affinity for the target protein in its naturally-occurring state The animal to be immunized is preferably an adult animal, and the cell is phenotypically similar or identical to the animal to be immunized. For example, the cell expressing the heterologous nucleic acid is obtained from the same animal. Alternatively, the cell is obtained from a different mature animal.

The animal is a member of an avian species, e.g., a chicken. Any breed of chicken is used to produce antibodies against a selected target antigen. Exemplary varieties include White Leghorn, Rhode Island Red, Plymouth Rock, Dominiques, Wyandottes, Rhode Island Reds, Rhode Island Whites, Buckeyes, Chanteclers, Jersey Giants, Lamonas, New Hampshires, and Delawares. Alternatively, the animal is a rodent, e.g., a member of a murine species such as a mouse, or a lagomorph, e.g., a rabbit.

The immunogen is an undifferentiated stem cell or a mature differentiated cell. Preferably, the cell does not express embryonic antigens, e.g., the cell is a not an embryonic cell. For example, the immunogen cell is derived or obtained from a mature animal. The cell is preferably a spleen cell or a bone marrow cell into which the heterologous nucleic acid has been introduced. Optionally, the cell is immortalized.

The nucleic acid is operatively linked to a ubiquitous or a tissue-specific promoter. A tissue-specific enhancer may also be used to augment expression in a preferred target tissue.

Also within the invention is a method of elucidating the function and subcellular location of a polypeptide encoded by the heterologous DNA.

The methods of the invention provide several advantages over previously-described methods for producing specific antibodies. For example, the use of transfected syngeneic cells (or an acellular product thereof) as an immunogen specifically targets the immune response against the antigen of interest. Another advantage is that expression of the gene sequence of interest in the syngeneic cell leads to expression of the target immunogenic protein in a physiologically relevant manner (e.g., the secondary and tertiary structure more closely resembles that of the naturally-occurring antigen compared to other immunization methods). For example, target antigen is presented to the immune system in its native configuration (e.g., secreted, membrane bound, lysosomal). Use of syngeneic cells as immunogens also generates a more rapid immune response compared to conventional immunization methods. Yet another advantage is that the transfected syngeneic cells are useful as a source of antigen and are therefore useful to monitor the production of a desired antibody by the immunized animal.

Other features and advantages of the invention will be apparent from the following description of the preferred embodiments thereof.

### BRIEF DESCRIPTION OF THE DRAWINGS

FIGS. 1A-B are photomicrographs of cells showing that sera from mice immunized with CD4-transfected cells contained CD-4 specific antibodies. FIG. 1A shows sera from mouse 1 in group 2 (g2 m1) incubated with cells transfected with CD4-encoding DNA (1p) FIG. 1B shows sera from the same mouse incubated with untransfected cells as a control (1n). The immune sera detected membrane-bound CD4 expressed on the surface of transfected cells. A CD4 signal was not detected on the surface of untransfected cells. The transfected cells shown in FIGS. 1A-B and 2A-B were confirmed positive for membrane expression of human CD4 using a commercially available CD4-specific antibody.

FIGS. 2A-B are photomicrographs of cells showing that sera from mice immunized with CD4-transfected cells contained CD4 specific antibodies. FIG. 2A shows sera from mouse 2 in group 2 (g2 m2) incubated with cells transfected with CD4-encoding DNA (1p) FIG. 2B shows sera from the same mouse incubated with untransfected cells as a control (1n). The immune sera detected membrane-bound CD4 expressed on the surface of transfected cells. A CD4 signal was not detected on the surface of untransfected cells.

### DETAILED DESCRIPTION

The immune system in higher animals is designed to protect against the invasion of foreign substance, and one of its primary methods of defense is the production of antibodies. For many years, researchers have studied the immune system to learn how it functions and to develop methods for controlling its actions. In this manner, researchers have been able to exploit the immune system of the human body to help fight disease, the immune system of other animals to generate antibodies for diagnostic and therapeutic use, and to develop drugs which can regulate its actions. The immune system has a well developed system for determining self from non-self, which has been demonstrated in experiments involving organ transplantation. Organ transplants between syngeneic animals such as inbred strains of mice are not rejected by the recipient's immune system, whereas allogeneic transplants are rejected. The underlying premise of the invention is to exploit the self/non-self recognition ability of the immune system to generate an antibody response to a specific antigen encoded by a DNA fragment, e.g. a nucleic acid encoding an uncharacterized protein such as those identified in genome sequencing efforts.

The sequencing of the human genome (Venter et al., 2001, Science 291:1304-1351) and development of rapid gene isolation techniques has identified thousands of genes with unknown function. To identify function and exploit the results of the genome sequencing effort, researchers have been attempting to develop a rapid technique for producing antibodies directly from an expressed nucleic acid molecule, e.g., a gene or cDNA. One technology known as DNA immunization involves the direct injection of the gene or cDNA under the control of a promoter directly into the animal to allow in vivo expression of the antigen (Ulivieri et al., 1996, J. Biotechnol. 51:191-4; and Yeung et al., 1997, J. Lipid Res. 38:2627-32. This technique has been shown to elicit an antibody response against several known proteins but has not been widely used for genomic derived or unknown genes. The earlier techniques are hampered by the lack of antigen for testing the immune response generated. The methods described herein overcome that problem, because the transfected syngeneic cells are useful not only as immunogens but as a source of antigen to screen and test for antibody production by the immunized animal as well as for screening hybridoma cells to identify monoclonal antibody-producing clones.

The method being described is applicable to all species, which produce antibodies (mammals as well as avian species), including but not limited to sheep, mice, goats, chickens, and rabbits. A critical factor is to use an immortalized cell line from the species or even sub-species being used for antibody production so that the cell line could be defined as syngeneic or genetically identical to the species of interest. Therefore, the cell by itself elicits little or no immune response when transferred back into the species of interest. Introduction of a novel gene, cDNA, or DNA fragment into the cell line would then represent a unique antigen which would be targeted by the immune system upon injection. The ability of the cell line to divide and express the antigen provides a constant stimulation for the immune system.

In addition to making antibodies specific for uncharacterized polypeptide antigens, the methods are useful to elucidating the function and subcellular location of the polypeptide encoded by the heterologous DNA. The antibodies produced following immunization with transfected syngeneic cells are used to characterize the polypeptide. For example, the antibodies bind to the membrane of a cell transfected with an uncharacterized heterologous DNA (and do not bind to the membrane of control untransfected cells), the data indicates that the uncharacterized antigen is a membrane polypeptide. Similar techniques are used to determine whether the antigen is expressed in other subcellular locations and to determine functional characteristics of the antigen.

Syngeneic Cell-Based Immunization

The invention provides methods for quickly moving from a gene or cDNA identified by genomics or rapid screening technologies to an antibody specific for the gene product of interest. Such antibodies are developed as therapeutic agents, used to determine the pattern of expression in cells or tissues, or used as a diagnostic reagent to identify aberrant patterns of gene expression associated with disease states.

Highly specific antibodies against unknown proteins are produced utilizing a DNA sequence such as a gene or cDNA. The production technique eliminates the need to have a source of antigen and provides a source of antigen to test for antibody production. The invention is useful to generate antibodies in any number of species such as a mouse, rabbit, chicken, sheep, goat, or cow and only requires the availability or development of a syngeneic immortalized cell line.

A nucleic acid, e.g., a DNA fragment encoding a target antigen, is introduced into the syngeneic immortalized cell line under the control of an appropriate promoter and selectable marker to allow expression. The transfected cells are then used the cells to immunize the animal. The use of a syngeneic cell line specifically targets the antibody response to the protein encoded by the gene of interest since all other proteins are viewed as self. This overcomes a major problem of conventional antibody production techniques in which antigens are delivered as fusion proteins (beta-gal) or as a mixture of many proteins (cell lysate/organ lysate/tumor lysate) generating an antibody response against many different antigens. In addition, this technique provides a source of antigen, i.e., the transfected immortalized syngeneic cells, for testing the immune response unlike gene based immunization technologies in which the gene is introduced directly into the animal.

The method includes the following steps: 1) Production of an immortalized cell line from a desired species, 2) Transfection of the cell line with the nucleic acid of interest linked to a promoter (if necessary) and selectable marker such as antibiotic resistance, 3) Selection of the transfected cells and expansion of the culture for cryopreservation and immunization, 4) immunization of the animal with live or dead cells to elicit an antibody response, and 5) collection of serum/egg yolk to test for an antibody response. The antibody response mounted by the immunized animal is evaluated using standard methods. In the case of a secreted protein, antibody production is assessed using the culture media from the transfected cells. In the case of non-secreted proteins, whole cell samples or cell lysates are tested for presence of an antibody specific for the target antigen. Non-transfected cells are used as a negative control to judge the specificity of the antibody response.

Polyclonal and Monoclonal Antibodies

The methods are used to produce polyclonal antisera as well as to generate cells useful in producing a monoclonal antibody. The term antibody encompasses not only an intact monoclonal antibody, but also an immunologically-active antibody fragment, e.g., a Fab or (Fab)₂ fragment; an engineered single chain Fv molecule; or a chimeric molecule, e.g., an antibody which contains the binding specificity of one antibody, e.g., of murine origin, and the remaining portions of another antibody, e.g., of human origin. An antibody that reacts with or is specific for a target antigen is an antibody that binds an epitope present on the antigen.

Monoclonal antibodies are obtained using antibody-producing cells obtained from mice or other animals immunized using transfected syngeneic cells (or acellular products thereof) as described above. Antibody-producing hybridomas are made using standard methods. Antibody-producing cells from the immunized animal are fused to a myeloma cell, e.g., the SP2/0 myeloma (GM3659 B, NIGMS Human Genetic Mutant Cell Repository, Camden, N.J.). The fusion is performed using well known protocols, e.g., Oi et al., 1980, “Immunoglobulin-producing hybrid cell lines” in Selected Methods in Cellular Immunology, Mishell and Shiigi, eds., W. H. Freeman and Co., San Francisco, pp. 357-362). For example, spleen cells are mixed with SP2/0 at a ratio of 5:1, and 50% polyethylene glycol 1500 is used as the fusagen. To identify those hybridomas producing antibodies that are highly specific for a target antigen, hybridomas are screened using the same syngeneic cell immunogen that was used to immunize the animals. Alternatively, the hybridomas are screened using purified target protein. The antibody preferably has a binding affinity of at least about 10⁸ liters/mole and more preferably, an affinity of at least about 10⁹ liters/mole.

Monoclonal antibodies are humanized by methods known in the art, e.g., MAbs with a desired binding specificity can be commercially humanized (Scotgene, Scotland; Oxford Molecular, Palo Alto, Calif.). Methods of producing monoclonal antibodies and methods of generating heterologous antibodies (e.g., human antibodies) in non-human animals are known (e.g., U.S. Pat. Nos. 5,874,299; 5,545,806; 5,569,825; 5,661,016; 5,625,126; 5,633,425; 5,770,429; and 5,789,650).

The following examples illustrate various aspects of the invention.

### EXAMPLE 1

### Production of antibodies in mice Anti-human CD4 antibodies were produced in mice as follows:

The human CD4 cDNA was cloned into the mammalian expression vector, pcDNA3 (Invitrogen, Carlsbad, Calif.). The vector contains a cytomegalovirus (CMV) promoter and neomycin resistance as the selectable marker. The vector was linearized with Pvu I, and transfected into C127i cells (ATCC, Manassas, Va.) using Lipofectamine/OptiMEM transfection system (Invitrogen, Carlsbad, Calif.). After 3 days, the cells were harvested, split into three wells of a 6 well tissue culture plate, and selected with G418 at 0.25 mg/ml, 0.5 mg/ml, or 1.0 mg/ml for 2 weeks. After 2 weeks of selection, the cells had reached 80% confluency and were scaled up for immunization of mice.

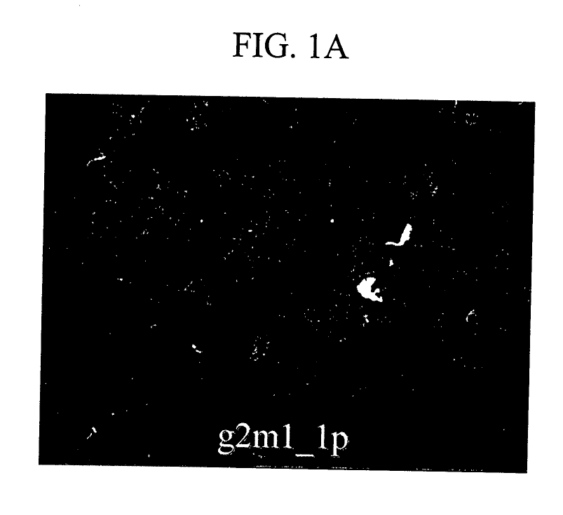

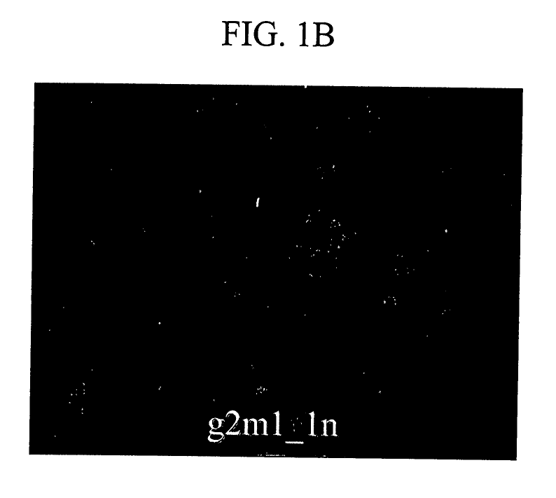

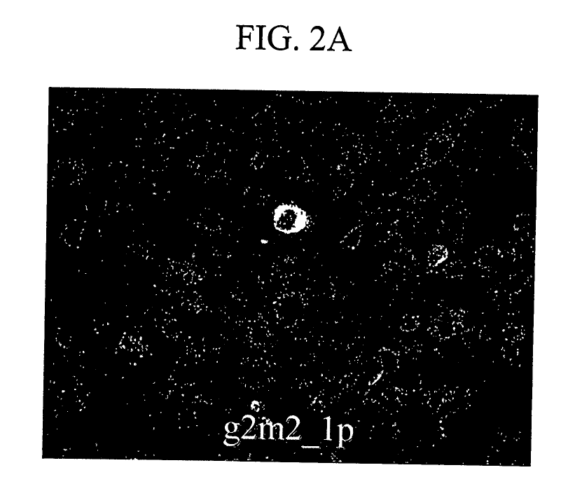

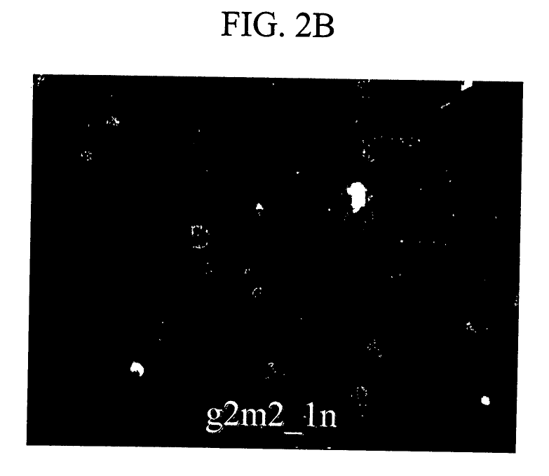

To test the ability of the CD4-C127 cells to elicit an antibody response to test target antigen, human CD4, whole live cells were used to immunize CD-1 mice. Two CD-1 mice were injected on day 0 with 0.1 cc of CD4-C127 cells (1×10⁵/ml) subcutaneously and 0.1 cc of RIBI adjuvant (Sigma Chemical, St Louis, Mo.) subcutaneously at two separate sites, day 14 with 0.1 cc of CD4-C127 cells subcutaneously, and intravenously on day 28 with CD4-C127 cells. Two weeks after the final injection, the mice were euthanized and serum collected to test for the presence of antibody. The presence of antibody was detect by incubating the serum with both non-transfected C127 cells and CD4-C127 followed by a goat anti-mouse IgG FITC-labeled secondary antibody, and visualization under ultraviolet light (FIGS. 1A-B and 2A-B). The presence of a strong green fluorescence under ultraviolet light indicates the presence of mouse antibodies in the serum. A comparison of the non-transfected and CD4-C127 cells shows a specific reaction to the human CD4 and a very weak reaction to the C127 cells. The weak diffuse signal is due to the reaction of the mouse immune system to some of the antigens on the C127 cells, since this cell line was derived from a balb/c mouse strain and not the CD-1, i.e., the immunogen cell was closely genetically matched but not genetically identical to the recipient animal.

### EXAMPLE 2

### Antibody Production in Rabbits

The methods described herein are used to produce antibodies in rabbits. Rabbit cell lines are commercially available. If an appropriate syngeneic rabbit cell line is unavailable, custom immortalized cells are made and transfected for use as immunogens.

Immortalization of a Cell Line:

A panel of four tissues (spleen, blood, kidney, liver) is harvested from a New Zealand white rabbit for development of an immortalized cell line. In the case of solid organs, the cells are dispersed by mincing the tissue with a scalpel, incubating the minced tissue at 37° C. for 1 hr with 0.3 mg/ml collagenase, 1% dispase in DMEM plus 10% fetal bovine, followed by repeated pipeting. The large pieces are removed and the dispersed cells collected and placed in culture in standard culture medium, e.g., DMEM plus 10% fetal bovine serum. With blood, the white blood cells are harvested by ficoll gradient centrifugation, washed with PBS, and placed in culture in DMEM plus 10% fetal bovine serum. The next day, the cultures are transfected with the plasmid pSV3neo (Weingartl et al., 2002, J. Virol. Methods 104(2):203-16) or treated with a carcinogen (Rhim et al., 1993, Crit. Rev. Oncop. 4:313-335.) and the cultures selected for rapidly dividing colonies. Rapidly dividing colonies are expanded and the cell lines cryopreserved in 10% DMSO in DMEM with liquid nitrogen.

Construction of an Expression Vector and Transfection:

To obtain high level expression of the desired antigen, the promoter and 3′flanking region of a ubiquitous promoter such as keratin is cloned from a genomic library or by the polymerase chain reaction from genomic DNA. Sequence of the keratin 7 gene was retrieved from Genbank and oligo nucleotides were designed to the promoter as follows: (KER7-1: 5′GTCGACATATGTTACAAACTAGC3′ (SEQ ID NO:1); KER7-3: 5′CTCGAGTTGGCCTCTGCCACAG3′ (SEQ ID NO:2)) and 3′flanking sequence (KER7-4: 5′CTCGAGTAGACTCACTGAGGCA3′(SEQ ID NO:5); KER7-5: 5′GCGGCCGCAGTTATTGTGGCCAAA3′ (SEQ ID NO:6)). Using the polymerase chain reaction the promoter and 3′ flanking region of the human keratin gene were cloned into the vector pCR2.1 (Invitrogen, Carlsbad, Calif.). The promoter was then ligated to the 3′ flanking region using the common XhoI site and cloned into a modified DsRed vector (Clontech, Palo Alto, Calif.) which contains a neomycin selectable marker. The new vector was designated pKER8. pKER8 is digested with XhoI to allow for the insertion of a 15 Kb SalI fragment containing the human serum albumin gene. The new vector is restriction mapped to confirm the correct orientation of the albumin gene and is designated pKER-HSA.

Production of an antigen expressing rabbit cell line:

The immortalized rabbit cell line is grown in a T25 tissue culture flask to 70-80% confluence. The cells are transfected with the SalI linearized pKER-HSA vector using the Lipofectamine/OptiMem system (Invitrogen, Carlsbad, Calif.). The cells are allowed to recovery for 48 hrs before being split 1 to 10 and placed under selection of G418 at 0.5-1.0 mg/ml. After the cells reached 80% confluency, the culture is expanded in order to cryopreserved the cell and immunize rabbits. Cells used for immunization are washed extensively with PBS to remove any contaminating proteins from the tissue culture media.

Immunization of Rabbits and Screening for Antibody Production:

Six New Zealand white rabbits approximately 3-4 Kg in weight are divided into three groups of two for immunization with the pKER-HSA cells. Each group of rabbits is immunized using a different protocol to determine the best route of delivery of antigen. The immunization protocol is outlined in the table below:

TABLE 1

| Group | Day 0 | Day 14 | Day 21 | Day 28 |
|---|---|---|---|---|
| 1 | SQ 1 × 10⁷ | SQ 1 × 10⁷ | IV 1 × 10⁷ | Collect Serum |
| | cells | cells | cells | |
| | SQ adjuvant | | | |
| 2 | IM 1 × 10⁷ | | | |
| 2 | IM 1 × 10⁷ | IM 1 × 10⁷ | IV 1 × 10⁷ | Collect Serum |
| | cells | cells | cells | |
| | SQ adjuvant | | | |
| 3 | IV 1 × 10⁷ | IV 1 × 10⁷ | IV 1 × 10⁷ | Collect Serum |
| | cells | cells | cells | |

SQ = subcutaneously,
IV = intravenously,
IM = intramuscularly

The serum collected from the three groups of rabbits is tested for the presence of antibody using a standard ELISA assay. A 96 well is coated at 4° C. with media from the pKER-HSA cell line mixed 1:1 with 0.1M sodium bicarbonate pH 9.2. The plated is washed with PBS/0.1% Tween 20 and 0.1 ml of serial dilutions of the six serums starting at 1:1 is added to the wells. The plate is incubated in a humidified environment at 37° C. for 1 hr, washed with PBS/0.1% Tween 20, and 0.1 ml of HRP-conjugated goat anti-rabbit antibody added at a 1:5000 dilution. Following 1 hr incubation at 37° C., the plate is washed with PBS/0.1% Tween 20, and a positive antibody reaction visualized by adding 0.1 ml of citric phosphate buffer containing 0.4 mg/ml o-phenylenediamine. The reaction is terminated after 30 minute by adding 50 μl of 4.5M sulfuric acid and the intensity of the color determined by absorbance at 490 nm using a 96well plate reader. All rabbits showing a positive antibody response are bled on a weekly schedule and given an IV boost as needed. Any rabbits not showing a positive antibody titer are given a second IV boost of pKER-HSA cells and retested.

### EXAMPLE 3

### Antibodies in Chickens

Unlike mammals that target antibodies to their milk to offer protection to their young, avian selectively target antibodies to the yolk of their eggs offering an easy collection system for harvesting the antibodies. In addition, the genetic difference between mammals and avians allowed for a stronger immune response to many of the antigens being targeted. This invention is easily adapted to the avian system because fertilized eggs offer a readily available source of both stem cells and differentiated cells which can be transfected with a target DNA and used as immunogen cells, thereby eliminating the need for immortalization of cells. For example, primary chicken embryonic (CEC) and fibroblast (CEF) cells have been transfected and maintained in culture for 1-3 months. These cells are transfected with nucleic acids encoding a target antigen and used as immunogens.

The cells and chicken recipient are genetically matched at MHC loci, e.g., class I and II loci. There are two loci with class I and class II genes in chickens, the B-F/B-L region of the B locus and the Rfp-Y locus, which are genetically unlinked. The B-F/B-L region (but not the Rfp-Y locus) classical class I and class II genes, which mediate responses to serological alloantigens, rapid allograft rejection, strong mixed lymphocyte reaction and cellular cooperation in immune responses.

Embryonic chicken cells are harvested from a Day 0 to 9 developing white leghorn egg, dispersed and placed in culture in DMEM with 10% fetal bovine serum. The next day, the cells are transfected with SalI linearized pKER-HSA using a BioRad electroporation system with 200V for 2 mSec duration, 100% modulation, 10 bursts, and a 1 sec burst interval. The embryonic cells are placed back in culture and selected with 0.5 to 1.0 mg/ml of G418 for two weeks. After two weeks, the culture is expanded and the cells cryopreserved and used to immunize 4-8 month old white leghorn hens. The chickens are immunized according to the following schedule:

TABLE 2

| Group | Day 0 | Day 14 | Day 21 | Day 28 |
|---|---|---|---|---|
| 1 | SQ 1 × 10⁷ | SQ 1 × 10⁷ | IV 1 × 10⁷ | Collect eggs |
| | cells | cells | cells | |
| | SQ adjuvant | | | |
| 2 | IM 1 × 10⁷ | IM 1 × 10⁷ | IV 1 × 10⁷ | Collect eggs |
| | cells | cells | cells | |
| | SQ adjuvant | | | |
| 3 | IV 1 × 10⁷ | IV 1 × 10⁷ | IV 1 × 10⁷ | Collect eggs |
| | cells | cells | cells | |

The eggs collected from the three groups of hens are tested for the presence of antibody using an elisa. A 96 well is coated at 4° C. with media from the pKER-HSA cell line mixed 1:1 with 0.1M sodium bicarbonate pH 9.2. The plated is washed with PBS/0.1% Tween 20 and 0.1 ml of serial dilutions of the six serums starting at 1:1 was added to the wells. The plate is incubated in a humidified environment at 37° C. for 1 hr, washed with PBS/0.1% Tween 20, and 0.1 ml of HRP-conjugated goat anti-chicken antibody added at a 1:5000 dilution. Following 1 hr incubation at 37° C., the plate was washed with PBS/0.1% Tween 20, and a positive antibody reaction visualized by adding 0.1 ml of citric phosphate buffer containing 0.4 mg/ml o-phenylenediamine. The reaction is terminated after 30 minute by adding 50 ul of 4.5M sulfuric acid and the intensity of the color determined by absorbance at 490 nm using a 96well plate reader. Eggs are collected daily from all chickens showing a positive antibody response and hens are given an IV boost as needed. Any hen not showing a positive antibody titer is given a second IV boost of pKER-HSA cells and retested.

Other embodiments are within the following claims.

What is claimed is:

1. A method of producing an antibody to a target antigen in an animal, comprising contacting said animal with a genetically-matched cell containing a heterologous nucleic acid, said nucleic acid encoding said target antigen.

2. The method of claim 1, wherein said animal and said cell are syngeneic at major histocompatibility complex (MHC) loci.

3. The method of claim 1, wherein said cell expresses a gene product encoded by said nucleic acid.

4. The method of claim 3, wherein said gene product is expressed on a surface of said cell.

5. The method of claim 1, wherein said animal is adult and said cell is obtained from the same or different adult animal.

6. The method of claim 1, wherein said animal is a member of an avian species.

7. The method of claim 6, wherein said animal is a chicken.

8. The method of claim 1, wherein said animal is a rodent.

9. The method of claim 1, wherein said animal is a member of a murine species.

10. The method of claim 9, wherein said animal is a mouse.

11. The method of claim 8, wherein said animal is a rabbit.

12. The method of claim 1, wherein said cell is an undifferentiated stem cell.

13. The method of claim 1, wherein said cell is a differentiated cell.

14. The method of claim 1, wherein said cell is a not an embryonic cell.

15. The method of claim 1, wherein said cell is derived from a mature animal.

16. The method of claim 1, wherein said cell is a spleen cell.

17. The method of claim 1, wherein said cell is a bone marrow cell.

18. The method of claim 1, wherein said cell is immortalized.


SEQUENCE LISTING (6 sequences)

SEQ ID NO: 1 - DNA, 23 bp, Artificial Sequence
  Description of Artificial Sequence:Oligonucleotide primer
gtcgacatatgttacaaactagc

SEQ ID NO: 2 - DNA, 22 bp, Artificial Sequence
  Description of Artificial Sequence:Oligonucleotide primer
ctcgagttggcctctgccacag

SEQ ID NO: 3-4 - skipped (2 entries)

SEQ ID NO: 5 - DNA, 22 bp, Artificial Sequence
  Description of Artificial Sequence:Oligonucleotide primer
ctcgagtagactcactgaggca

SEQ ID NO: 6 - DNA, 24 bp, Artificial Sequence
  Description of Artificial Sequence:Oligonucleotide primer
gcggccgcagttattgtggccaaa